SEQUENCE LISTING (2 sequences)

SEQ ID NO: 1 - DNA, 32 bp, Artificial Sequence
  Description of Artificial Sequence Primers
ccgtgctagcccagaatgaaggctgggacagc

SEQ ID NO: 2 - DNA, 34 bp, Artificial Sequence
  Description of Artificial Sequence Primers
ctattggatcccgcccagtgtcaggggcggactg

(19) United States
(12) Patent Application Publication
(10) Pub. No.: US 2004/0202675 A1
(43) Pub. Date: Oct. 14, 2004

(54) Vaccine adjuvant

(75) Inventor: Barton F. Haynes, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC (US)

(21) Appl. No.: 10/730,379

(22) Filed: Dec. 9, 2003

Related U.S. Application Data
(60) Provisional application No. 60/431,727, filed on Dec. 9, 2002.

(51) Int. Cl. A61K 48/00; A61K 39/00

(52) U.S. Cl. 424/185.1; 514/44

(57) ABSTRACT

The present invention relates generally to K12 and more specifically to the use of K12 as a non-antigen specific activator of immune function.

This application claims priority from Provisional Application. No. 60/431,727, filed Dec. 9, 2002, the content of which is incorporated herein by reference.

### TECHNICAL FIELD

The present invention relates generally to K12 and more specifically to the use of K12 as a non-antigen specific activator of immune function.

### BACKGROUND

CD7 is a 40 kD immunoglobulin gene superfamily molecule expressed on T and natural killer (NK) cells and on T, B and myeloid cell precursors (Crit. Review Immunology 19:331-348 (1999)). Functional characterization of CD7 has been slowed by the lack of availability of a CD7 monoclonal antibody (mab) against murine CD7. On human TCRαβ T cells, CD7 mabs serve as weak co-mitogens with submitogenic amounts of CD3 mabs (Cell. Immunol. 14:189-199 (1992)). However, TCRγδ crosslinking of CD7 mabs bound to CD7 on T cells induces a Ca²⁺ flux, TCRγδ cell production of IL-2, and TCRγδ cell proliferation (Eur. J. Immunol. 21:1195-1200 (1991)). Ligation of CD7 on natural killer (NK) cells also induces cytokine (IFN-γ) production and NK cell proliferation (J. Immunol. 152:517 (1994)). CD7 knockout mice have normal TCRαβ cell maturation but have diminished populations of NK/T cells in the liver, and are resistant to lipopolysacharride (LPS) induced shock (J. Exp. Med. 189:1011-1016 (1999)). Thus, CD7 is a key signaling molecule for TCRαβ, TCRγδ and NK cells. CD7 also plays a role in regulation of TCRγδ and NK cytokine production and T/NK cell ontogeny.

The principal ligand for CD7 has been found to be a secreted molecule, K12, also an Ig superfamily molecule (Genomics 47:327-340 (1998); J. Biol. Chem. 275:3431-3437 (2000), U.S. Pat. No. 6,350,615, US 20020141999). Interestingly, the K12 gene is located immediately 5′ to the CD7 gene and the K12-CD7 receptor ligand pair is the only known such pair the genes for which are immediately adjacent in the genome. K12 was originally found to be produced by epithelial cell lines as well as granulocytes but not by lymphocytes (Szent-Kestler, Genomics 47:327-340 (1998)). However, CD7 deficiency combined with CD28 deficiency led to decreased thymocyte IFN-γ and TNF-α production, and a decrease in CD25+ and B7-1 and B7-2 expressing cells in the thymus (Heinley et al, Intemat. Immunology 13:157-166 (2001)). While K12 mRNA expression in human thymus has been reported (Szent-Kestler, Genomics 47:327-340 (1998)), the thymic cell types that produce K12 were not identified.

The present invention relates to a method treating infection in a patient that takes advantage of the ability of K12 to function as a non-antigen specific activator of the patient's innate immune system. Moreover, the present invention relates to a method of providing a novel adjuvant system for enhancing immune responses to vaccines.

### SUMMARY OF THE INVENTION

The present invention relates generally to K12 and more specifically to the use of K12 as a non-antigen specific activator of immune function, for example, as a treatment of infectious diseases and of cancer and as an adjuvant to enhance vaccine responses.

Objects and advantages of the present invention will be clear from the description that follows.

### BRIEF DESCRIPTION OF THE DRAWINGS

FIG. 1. CD7, K12, and control (CD8) fusion protein constructs. cDNA clones encoding CD7 and K12 were ligated with human IgG₁ constant region genomic sequence, and subsequently inserted into CDM8 plasmid to allow the segment encoding the CD7 and K12 domains to be joined at restriction enzyme sites Nhe and BamH1 with human IgG₁. hCD8-Ig fusion protein was used as a control in selected experiments.

FIGS. 2A and 2B. FIG. 2A. Dose-dependent binding of soluble CD7 versus soluble CD8 control to immobilized ligand K12. Soluble hCD7-Ig fusion protein bound specifically to immobilized hK12-Ig fusion protein with increasing response at 6.25 μg/mL, 12.5 μg/mL, 25 μg/mL, 50 μg/mL, and 100 μg/mL of soluble protein, as determined by biosensor-based technology of BIAcore 3000. Soluble hCD8-Ig fusion protein did not bind to K12. FIG. 2B. Kinetic analysis of the binding between soluble hCD7-Ig and hKl2-Ig. CD7 bound strongly to K12 to form a stable complex with a K_{eq} of 37.6×10⁻⁹ M.

FIG. 3. Identification of two mab's, K12-A1 and K12-H9, that inhibit K1 fusion protein binding to CD7 fusion protein.

FIGS. 4A and 4B. FIG. 4A. K12 mRNA is expressed by human primary (1°) TE, TF cells and the human TE cell line, TE750, as determined by RT-PCR and Northern blot analyses. Top panel demonstrates the negative of an ethidium bromide stained gel of RT-PCR products (˜400 bp). The left-hand arrow points to thepredominant band fof TE, TF, and TE750 cells and the right-hand arrow points to the product from a K12-containing plasmid. Bottom panel demonstrates a Southern blot of PCR products with the NheI/BamHI DNA fragment from hK12 extracellular domain. Hybridization of this fragment indicates the identity of the 400 bp band as hK12. FIG. 2B. K12 expression is upregulated by IFN-γ in thymic epithelial cells as detected by Northern blot analyses. Administration of 500 U/mL of IFN-γ to TE750 cells for 24 h dramatically upregulates mRNA expression of K12.

FIGS. 5A-5D. K12 expression in TE 750 cells treated with brefeldin A and IFN-γ by flow cytometry. TE cells from the TE750 cell line were isolated and treated with IFN-γ (500 U/mL) for 24 h and Brefeldin-A (1 μg/mL) for 4 h. Cells were incubated with saturating amounts of monoclonal antibody (mAb) against K-12 (solid lines) or with control mouse IgG_{2b} (dotted lines). In the absence of treatment (FIG. 5A) and with IFN-γ treatment alone (FIG. 5B), K-12 expression was not detected on the cell surface or intracellularly. However, with brefeldin A treatment (FIG. 5C), a slight increase in K-12 expression was detected, and in the presence of both brefeldin A and IFN-γ, an 45% upregulation of K-12 expression was observed (FIG. 5D). Brefeldin A inhibits protein transport by localizing synthesized protein to the Golgi complex.

FIGS. 6A-6F. K-12 is expressed in human thymic epithelial (TE) cells as detected by immunofluorescent staining with anti-K-12 monoclonal antibody, K12-A1 (FIGS. 6A-E) and human CD7-Ig fusion protein (FIG. 6F). K-12 was detected intracellularly in primary TE cells cultured in media alone for 48 h (FIG. 6A) and demonstrated a faint perinuclear, Golgi-like pattern of expression in a subset of TE cells. Treatment with IFN-γ (500 U/mL for 48 h.) upregulated K-12 expression (FIG. 6B). A similar staining pattern was observed in cells from the TE750 cell line treated with Brefeldin A, an inhibitor of protein transport that localizes synthesized protein to the Golgi complex (FIG. 6C). Treatment with IFN-γ dramatically upregulated K-12 expression. Magnification of this pattern to 400× (FIG. 6E) illustrates the perinuclear, Golgi-associated pattern of K-12 expression. An identical staining pattern is seen with human CD7-Ig fuision protein (FIG. 6F).

FIGS. 7A-7C. Detection of soluble K12 from 20×supernatant of TE 750 cells treated with IFN-γ (500 U/mL) for 72 h. BIAcore analysis of the dissociative phase of analyte flow demonstrates the differences in amount of soluble K12 that bound to both K12 A-1 mab and hCD7-Ig fusion protein versus hCD8-Ig (control) which were immobilized to the biosensor chip (FIG. 7A). Soluble K12 from TE750 cells treated with IFN-γ (500 U/mL) was detected in increasing amounts by K12 mab immobilized to a biosensor chip compared to untreated TE750 cells and media alone (control), as detected by BlAcore analysis (FIGS. 7B and 7C). IFN-γ treated: RU=324+19.0; untreated: RU=239+25.54; medium alone: RU=19+9.67. T-Test results: IFN-γ vs. untreated: p=0.022; IFN-γ vs. medium alone: p=0.008; untreated vs. medium alone: p=0.025.

FIG. 8. Blocking of soluble K12 binding to CD7-Ig with K12 A1 mAb. The interaction between soluble K12 from human TE750 cells was specifically and completely blocked with the addition of K12 A-1 mab.

FIGS. 9A-9E. Expression of K12 in human TE cells in situ in thymus is regulated by IFN-γ K12-A1 mab was reacted with sections of thymus explants at 0 hours (not shown), 6 hrs., 18 hrs. and 24 hrs. following treatment with 500 IU/mL IFN-γ. It was found that TE cells in fresh thymus tissue and TE cells in thymus tissue 6 hours (FIG. 9A) after treatment with IFN-γ was not reactive with the K12-A1 mab. FIG. 9B shows the negative control mab P3X63Ag8. However, after 18 (FIG. 9C) and 24 hrs (FIG. 9E) in IFN-γ 500 IU/mL, TE cells became positive for K12 reactivity. FIG. 9D shows reactivity of 18 hour treated thymus with anti-keratin mab AE1/AE-3. FIG. 9F shows 24 hour thymus treated with control media and negative for K-12 reactivity. All panels X400.

### DETAILED DESCRIPTION

CD7 triggering of TCRγδ cells and NK cells by CD7 ligand (K-12) is analogous to B7-1 and B7-2 triggering of TCRαβ cells, in that cross-linking of CD7 on NK and TCRγδ cells induces cell activation and triggering, whereas CD7 ligation on TCRαβ cells does not have this effect (Carrell et al, Eur. J. Immunol. 21:1195-1200 (1991)). The present invention relates to the use of soluble K12, or mimetic thereof, as an augmenter of a vaccine-induced immune response. In accordance with the invention, soluble K12, or mimetic thereof, serves as an adjuvant by stimulating production of TNF-α, IL-2 and IFN-γ by TCRγδ cells and NK cells, thereby activating and driving to differentiation T helper and T cytotoxic T cells. The invention further relates to the use of soluble K12, or mimetic thereof, as a non-specific stimulator of human T and NK cell function to treat, for example, bacterial, viral or fungal infections and malignancies. In this regard, soluble K-12, or mimetic thereof, can act to stimulate the innate immune system (TCRγδ and NK cells) to produce inflammatory cytokines that assist in controlling infectious and malignant diseases.

The term K12 as used herein includes the protein described in the publications cited herein (see particularly U.S. Pat. No. 6,350,615 and US 20020141999). As described in US 20020141999, the human K12 protein includes a signal sequence, an extracellular domain sequence, a transmembrane domain sequence, and a cytoplasmic domain sequence. The approximate boundaries for each region are given in US 20020141999. Soluble K12 lacks the functional transmembrane domain of the protein and can include the entire extracellular domain of human K12 or portion of the extracellular domain that retains binding affinity for CD7 (e.g., the extracellular domain can be truncated by up to about 20 amino acids at the N- and/or C-terminus). Homologous sequences are also suitable for use in the invention, such sequences include those at least 35% homologous to the extracellular domain of human K12 (see U.S. Pat. No. 6,350,615 and US 20020141999), or CD7 binding portion thereof, preferably, at least about 55% homologous, more preferably, at least about 70%, or 85% homologous, even more preferably at least about 95% homologous, and most preferably at least about 99% homologous, as determined by, for example, BLAST software, and that maintain a binding affinity for a CD7 protein. Also suitable for use in the invention are allelic variations of the human K12 extracellular domain, or CD7-binding portions thereof. As described in US 20020141999, naturally occurring soluble forms of K12 include fragments of approximately 19-22 Kd.

Specific examples of soluble K12 proteins are described in US 20020141999 and include approximately amino acid residues 49 to 125 and 49 to 165 of SEQ ID NO:4 of US 20020141999.

Soluble forms of K12 suitable for use in the present methods (and homologs thereof) can take the form of fuision proteins. That is, the soluble K12 sequence can be covalently linked to one or more heterologous amino acid sequence. Preferred heterologous amino acid sequences include sequences that promote interaction with cells of the immune system involved in antigen presentation, such as, the Fc portion of human IgG, C3d, alpha-2-macroglobulin or heat shock protein (e.g., heat shock protein 60).

In addition to the above, the present methods can also be practiced using mimetics of soluble K12. The mimetic can be, for example, a small molecule mimic of a soluble K12, or CD7-binding portion thereof. The mimetic can have increased stability, efficacy, potency and/or bioavailability, relative to soluble K12. Further, the mimetic can have decreased toxicity. The mimetic can be a peptidomimetic that includes L- ,D-, DL- or unnatural amino acids, alpha, alpha-disubstituted amino acids, N-alkyl amino acids, lactic acid (an isoelectronic analog of alanine). The peptide backbone of the peptidomimetic can have at least one bond replaced with PSI—[CH═CH]. The peptidomimetic can further include trifluorotyrosine, p-Cl-phenylalanine, p-Br-phenylalanine, poly-L-propargylglycine, poly-D,L-allyl glycine, or poly-L-allyl glycine. Examples of suitable unnatural amino acids include beta-alanine, L-alpha-amino butyric acid, L-Y-amino butyric acid, L-alpha-amino isobutyric acid, L-ε-amino caproic acid, 7-amino heptanoic acid, N-ε-Boc-N-alpha-CBZ-L-lysine, N-ε-Boc-N-alpha-Fmoc-L-lysine, L-methionine sulfone, L-norleucine, L-norvaline, N-alpha-Boc-N-delta-CBZ-L-omithine, N-delta-Boc-N-alpha-CBZ-L-omithine, Boc-p-nitro-L-phenylalanine, Boc-hydroxyproline, Boc-L-thioproline.

When the methods of the invention are practiced using soluble K12 (or fusion protein comprising same), the protein itself can be administered or a nucleic acid sequence that encodes the protein can be administered under conditions such that the nucleic acid sequence is expressed and the protein produced. The nucleic acid sequence can be introduced directly into the recipient subject, such as by standard intramuscular or intradermal injection, transdermal particle delivery, inhalation, topically, or by oral, intranasal or mucosal modes of administration. The nucleic acid sequence can be introduced ex vivo into cells (e.g., peripheral blood cells or lymph node cells) that have been removed from a subject and then cells containing the nucleic acid sequence re-introduced into the subject.

The nucleic acid sequence can be present in a vector that includes control sequences (e.g., a promoter) operably linked the nucleic acid sequence. Typical promoters for mammalian cell expression include the SV40 early promoter, a CMV promoter such as the CMV immediate early promoter, the mouse mammary tumor virus LTR promoter, the adenovirus major late promoter (Ad MLP), and other suitably efficient promoter systems. Nonviral promoters, such as a promoter derived from the murine metallothionein gene, can also be used for mammalian expression. Inducible, repressible or otherwise controllable promoters can be used. Typically, transcription termination and polyadenylation sequences are also be present in operable linkage. Enhancer elements can be included in order to increase expression levels. Examples of suitable enhancers include the SV40 early gene enhancer, the enhancer/promoter derived from the long terminal repeat (LTR) of the Rous Sarcoma Virus, and elements derived from human or murine CMV, for example, elements included in the CMV intron A sequence.

Vectors suitable for use in the invention include plasmid and viral vectors. Suitable viral vectors include modified vaccinia ankara, as well other vaccinia, recombinant Bacille Calmette-Guein and MTB. It will be appreciated that soluble K12 (including fusion proteins comprising same) can be produced chemically or recombinantly using standard techniques. (Soluble K12 and an antigenic component can also be expressed together or separately.)

Soluble K12 (including fusion proteins) and mimetics thereof can be formulated into compositions that include one or more excipients, diluents or carriers (e.g., squalene or other oil). When used as a vaccine adjuvant, soluble K12, or mimetic thereof, can be conjugated to the antigenic component of the vaccine or admixed therewith.

When soluble K12, or mimetic thereof, is used as a stimulant of the innate immune system, it can be used to defend against any of a myriad of infectious agents, including pathogens such as viriola major, the etiologic agent of small pox, Bacillus anthracis, the etiologic agent of anthrax, disseminated vaccinia, Yersinia pestis, the etilogic agent of bubonic plague which acts by suppressing human inflammatory responses. Y. pestis has recently been reported to suppress K-12 mRNA production in infected polymorphonuclear cells (Weissman et al, Blood 97: 2457-2468 (2001)). Early administration of soluble K-12, or mimetic thereof, (e.g., either orally or IM or IV) can enhance production of inflammatory cytokines that assist in controlling the infection, and thereby prevent serious disease. When soluble K12, or mimetic thereof, is used as an adjuvant for administration with vaccines, K12 can be either mixed with the vaccine, or directly conjugated to the vaccine, either by direct covalent coupling, coupling via a heterobifunctional agent (such as DTSSP) or by expression of K12 as a fusion gene or gene product with the gene or gene product of the vaccine protein.

Optimum dosing regimens can be readily established by one skilled in the art and can vary with the soluble K12 or mimetic thereof, the patient and the effect sought.

Certain aspects of the invention can be described in greater detail in the non-limiting Example that follows.

### EXAMPLE

Experimental Details

Cell culture. TE750 thymic epithelial cells were cultured as previously described (Miralles et al, Journal ofImmunology 160(7):3290-8 (1998)) and maintained at 37° C. in a fully humidified incubator. For experiments, cells were cultured in DME medium (Gibco, Grand Island, N.Y.) supplemented with insulin (5 μg/mL; Sigma, St. Louis, Mo.), adenine (1.8×10⁻⁴M; Sigma), sodium pyruvate (1.1×10⁻⁴M; Sigma), EGF (11.2 ng/mL; Collaborative, city, state), Amphotericin B (0.247 μg/mL; Gibco), and Gentamicin (50 μg/mL; BioWhitaker, Walkersville, Md.). In experiments where interferon-gamma (IFN-γ) (Chemicon Lot #19102706, Temecula, Calif.) was used, cells were washed×3 with PBS and treated with 200-500U/mL IFN-γ in DME medium. In experiments where brefeldin A (Beckton-Dickenson, Franklin Lakes, N.J.) was used, cells were washed×3 with PBS and treated with brefeldin A 1 μg/mL×4 h (37° C.) as per manufacturer's recommendations.

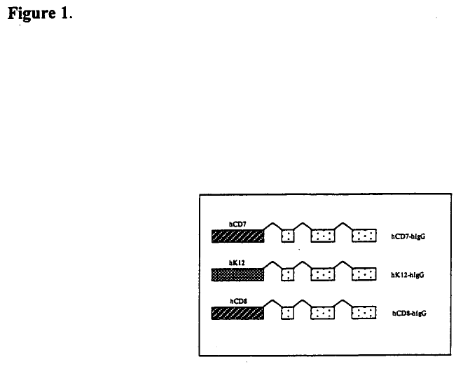

Generation of human CD7-Ig, K12-Ig, and CD8-Ig (control) fusion proteins (FIG. 1). CD8-Ig fusion protein construct was provided by Brian Seed (Massachusetts General Hospital, Boston, Mass.). Human CD7 and K12 expression plasmids were constructed by amplifying cDNA sequences by PCR using synthetic oligonucleotides complementary to sequences flanking the cDNA regions of CD7 and K12, respectively. Oligonucleotides were designed with the creation of restriction enzyme cleavage sites at the 5′ and 3′ ends of each amplified cDNA region so that subsequent insertion into human IgG₁ expression vectors could be facilitated. PCR amplification of K12 cDNA consisted of 25 cycles conducted at 94° C. for 30 s, 55° C. for 1 minute, and 72° C. for one minute using the reaction buffer recommended by the enzyme vendors (Gibco).

K12 primers contained of the following sequences:

5′-CCGTGCTAGCCCAGAATGAAGGCTGGGACAGC-3′
3′-GTCAGGCGGGGACTGTGACCCGCCCTAGGTTATC-5′

cDNA clones encoding CD7 (CD7 clone from Brian Seed, Harvard University, Boston, Mass.) and K12 were ligated with human IgG₁ constant region genomic sequence, and subsequently inserted into CDM8 plasmid (provided by Brian Seed, Massachusetts General Hospital, Boston, Mass.) to allow the segment encoding the CD7 and K12 domains to be joined at restriction enzyme sites Nhe and BamH1 with human IgG₁.

Plasmids containing hCD7-Ig and hK12-Ig were transfected into COS cells using DEAE dextran as previously described (Seed et al, EMBO Journal. 6(11):3313-6 (1987)). Typically, ten 100 mm semiconfluent plates of COS cells were transfected with each construct. Twelve hours after transfection, cells were trypsinized, seeded onto fresh plates with media (10% FCS) and incubated overnight. Transfected cells were allowed to grow in serum-free media for 4 days. On the fourth day, supernatants were harvested, centrifuged to remove non-adherent cells and debris, pooled, and stored (4° C.).

Purification of CD7-Ig and K12-Ig fusion proteins was performed via affinity chromatography with anti-hIg-agarose (Sigma) chromatography columns (BioRad, Melville, N.Y.). Elution of the proteins from the columns was performed using 100 mM glycine pH 3.0. Once isolated, proteins were concentrated and resuspended in phosphate-buffered saline (PBS) at concentrations of approximately 1 mg/mL.

BIAcore surface plasmon resonance (SPR) analyses. SPR analyses were performed as previously described (Alam et al., 1996). Briefly, 500-5000 resonance units (RU) of CD8-Rg, CD7-Ig or K12-Ig complexes were coupled to a CM5 sensor chip in 10 mM acetate buffer (pH 5.0), using the standard amine coupling technique. Binding of analytes was monitored in a BlAcore 3000 (BIAcore, Piscataway, N.J.) after equilibrating the instrument at the appropriate temperature. Analyte proteins were preincubated at the same temperature and then injected at a flow rate of 5-50 μL/min. Flow buffer used was PBS maintained at the same temperature as the instrument. Rate constant measurements and curve-fitting analyses were performed with the programs SPRevolution (Biotechnology Research Institute, National Research Council, Canada) and BIAevaluation 3.0 (BIAcore).

Preparation of anti-K12 monoclonal antibody (mab), K12-A1. Monoclonal antibodies to human K12 were prepared by standard techniques. Briefly, BALB/c mice (Charles River Laboratories, Inc., Wilmington, Mass.) were hyperimmunized with human K12-Ig fusion protein on days 0, 14, and 28. Splenocytes were fused with the murine myeloma cell line P3X63/Ag8 4 days after the last immunization, and hybrids were selected by aminopterin resistance. Hybridoma supernatants from approximately 1000 pools were screened for inhibition of human K12 by ELISA assay. Two mab, K12-A1 and K12-H9, were identified in this screen, cloned twice by limiting dilution, and further characterized. K12-A1 isotype is IgG2b, K.

Immunohistochemical analyses. TE750 cells treated with IFN-γ at 500 U/mL in media or with media alone were plated into 2-chamber Lab-Tek glass slides (Nunc, Napersville, Ill.) at a density of 10,000 cells/mL/well. K12-A1 mab supernatant was added to each chamber and incubated for 30 minutes at room temperature. Cells were then washed three times in PBS at 4° C. Subsequently, FITC-conjugated goat-anti-mouse Ig antibody (Kirkegaard and Perry Laboratories, Gaithersburg, Md.) was titrated for maximal fluorescence intensity and incubated with the cells for 30 minutes at room temperature. Cells were washed three times in PBS at 4° C. and analyzed by fluorescent microscopy with an Olympus Provis AX70 microscope.

Thymic epithelial (TE) activation andflow cytometry. TE750 cells treated with IFN-γ or media alone, and with brefeldin A or media alone were washed in media and counted on a Coulter Counter (Coulter Electronics, Hialeah, Fla.). For intracellular staining, 1×10⁶ cells were treated with 0.05% Tween 20 in FACS LYSE (Beckton Dickenson) for 1 h at room temperature. Cells were washed×3 (PBS) and incubated with saturating amounts of K12-A1 mab (1 h; RT). Cells were washed×3 and resuspended with FITC-conjugated goat-anti mouse Ig (1:10×1 h; RT). Samples were analyzed using a FACStar^{Plus} flow cytometer (Becton-Dickenson, Mountain View, Calif.). Specifically, 5000 viable events were analyzed using a forward and side scatter gate in CellQuest softward (Becton Dickenson).

Results

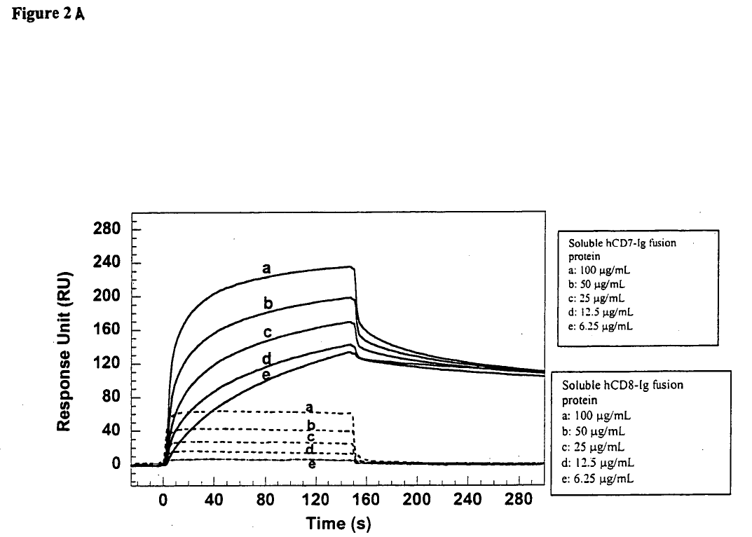

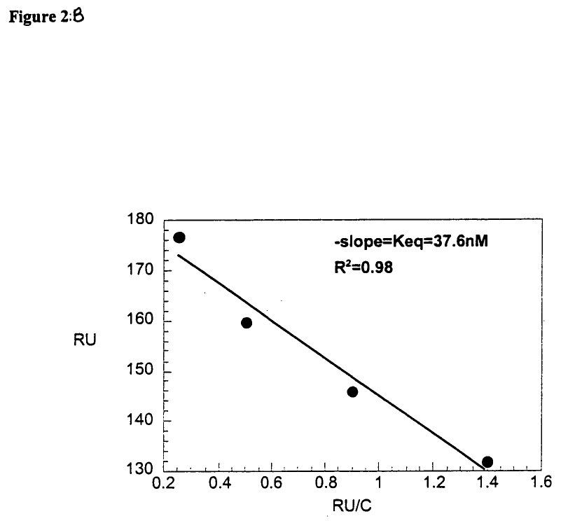

K12 is a ligandfor CD7. Lyman et al reported that recombinant human K12 was a ligand for human CD7 (J. Biol. Chem. 275:3431-3437 (2000)). Thus, recombinant human K12-Ig fusion protein has been studied and a determination made as to the ability of the fusion protein to bind to recombinant hCD7-Ig in surface plasmon reasonance (Biacore) assays. FIG. 2A shows the dose dependent binding of hCD7-Ig compared to hCD8-Ig to immobilized hK12-Ig. Whereas hCD8-Ig did not bind to K12 (dotted lines), hCD7-Ig bound specifically to immobilized hK12-Ig in a dose dependent manner (solid lines). Kinetic analysis of the binding between hCD7-Ig and hK12-Ig demonstrated a Keq of binding of CD7 to K12 of 38×10-9 M (FIG. 2B).

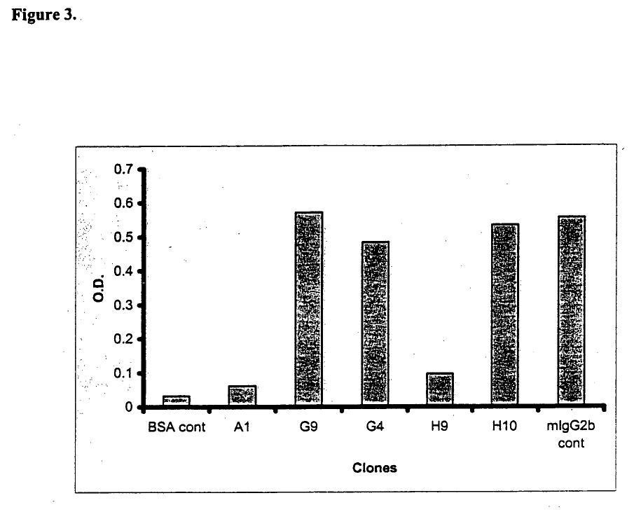

Development of monoclonal antibodies against human K12. To make reagents for characterizing human K12, mice were immunized with recombinant hK12-Ig and screened for reactivity of mabs with hK12-Ig that did not react with other Ig-bearing proteins. 5 anti-K12 mabs were identified, K12-A1, K12-G9, K12-G4, K12-H9 and K12-H10. In blocking assays of K12-Ig binding to CD7-Ig proteins in ELISA, two mabs were found that blocked K12-CD7 binding, K12-A1 and K12-H9 (FIG. 3).

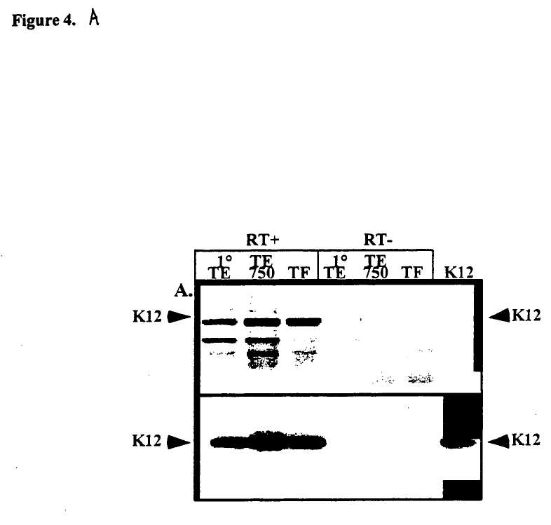

Expression of K12 in human thymic epithelial cells (TE). A determination was then made of the expression of hK12 mRNA in thymic fibroblasts and human TE cells either from primary TE cultures or the transformed TE cell line, TE 750 (FIG. 4). It was found that K12 mRNA was expressed by human primary TE cells, the human TE cell line TE750, and primary thymic fibroblasts (FIG. 4A, upper panel). Southern blot of PCR products with the nheI/BamH1 DNA fragment from hK12 extracellular domain demonstrated the identity of the 400 bp band as hK12 (FIG. 4A, lower panel). Northern blot analyses were performed on mRNA isolated from TE 750 cells, both before and after TE cell treatment with 500 IU/mL of IFN-g×24 hr. As has been shown with breast adenocarcinoma cells (Genomics 47:327-340 (1998)), IFN-γ treatment of human thymic epithelial cells markedly upregulated K12 mRNA expression in TE 750 cells (FIG. 4B).

To determine protein expression of K12 by cultured human TE cells, three methods were used: intracellular staining by flow cytometry and indirect IF, indirect IF on TE cells grown on Lab-Tek tissue culture slides, and analysis of secreted K12 in tissue culture supernatants.

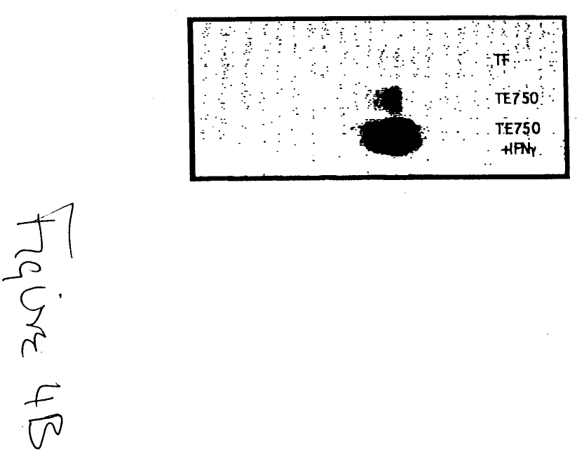

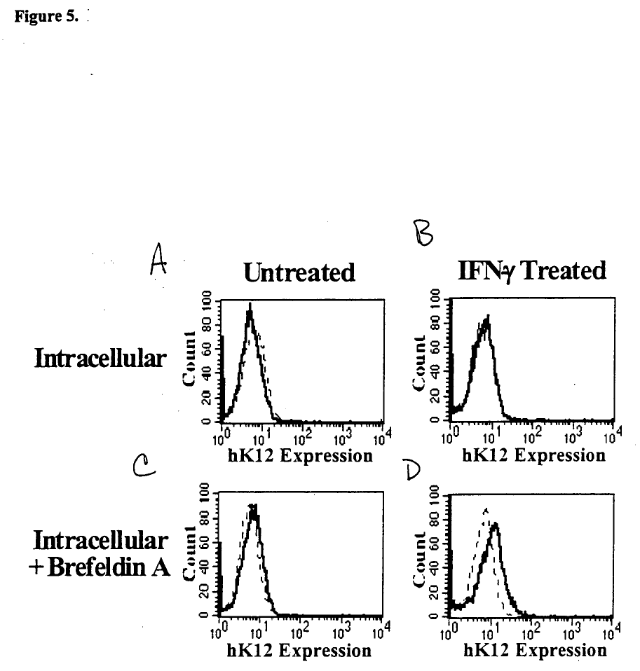

First, a determination was made of the ability of K12-A1 to detect constitutive or IFN-γ induced K12 expression of intracytoplasmic K12. TE 750 cells were fixed with acid alcohol and then reacted with saturating amounts of K12-A1 followed by goat anti-mouse Ig-FITC. Using this protocol, neither untreated nor IFN-γ-treated TE cells expressed intracellular K12 (FIGS. 5A and 5B). However, pretreatment of IFN-γ-treated TE 750 cells with 1 μg/ml of brefeldin A (protein transport inhibitor that localizes synthesized protein to the golgi complex), demonstrated a 45% upregulation of intracytoplasmic K12 (FIG. 5D).

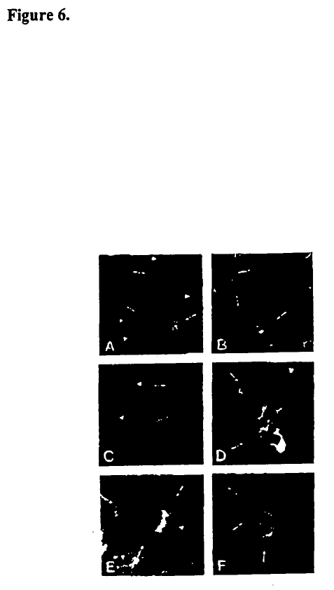

FIG. 6 shows indirect immunofluoresence assays on primary TE cells and the TE 750 cell line grown in culture on Lab-Tek slides in the presence or absence of IFN-γ. FIG. 6A shows scattered TE 812 primary culture of TE cells seeded on Lab-Tek slides in media×48 hrs. and then reacted with the K12-A1 mab in indirect IF. Arrows indicate several faint to moderately positive TE cells expressing K12, while arrowheads point to K12 negative TE cells. In contrast, TE 812 cells treated with IFN-γ 500 IU/mL×48 hrs. were all K12 positive in a golgi pattern (FIG. 6B). FIG. 6D shows the intense level of reactivity of the K12-A1 mab with TE 750 cells (arrows) that had been incubated×48 hrs. with IFN-γ as well as 1 μg/mL of brefeldin A, compared to the reactivity of K12-A1 mab with TE 750 cells incubated only with brefeldin A. FIG. 6E shows IFN-γ treated TE 750 cells reacting with K12-A1 mab in a golgi complex pattern, and FIG. 6F shows a similar pattern of intracellular K12 expression in IFN-γ treated TE 750 cells reacting with hCD7-Ig followed by anti-Ig-FITC.

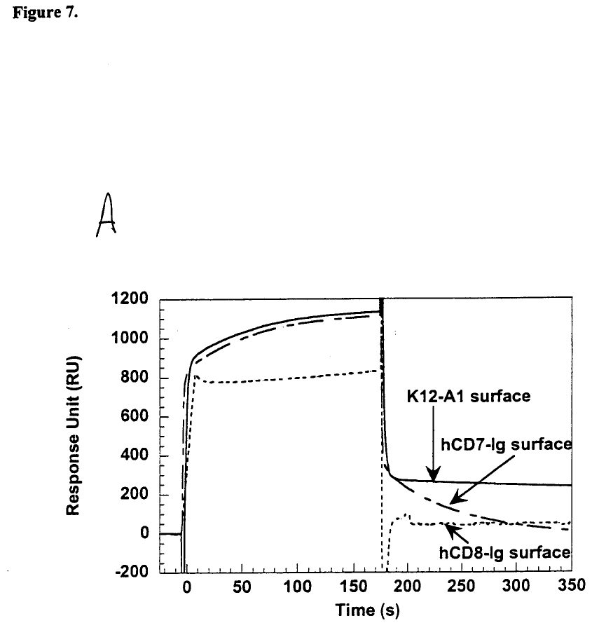

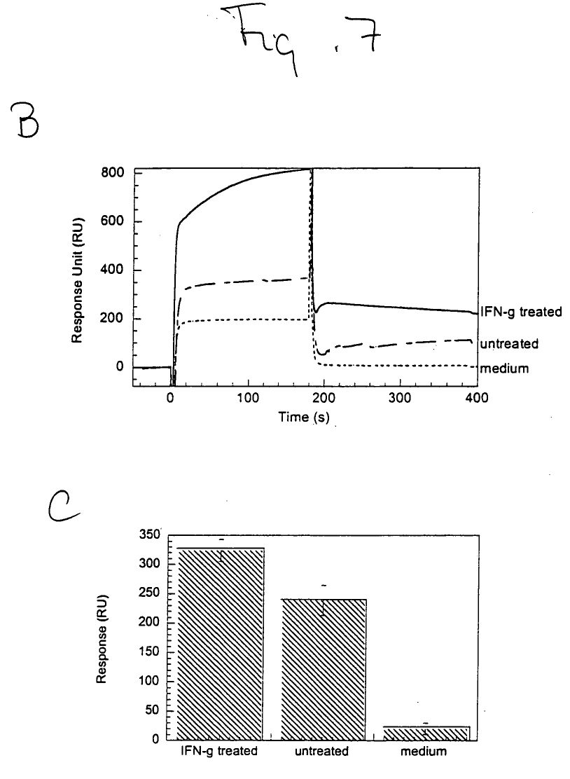

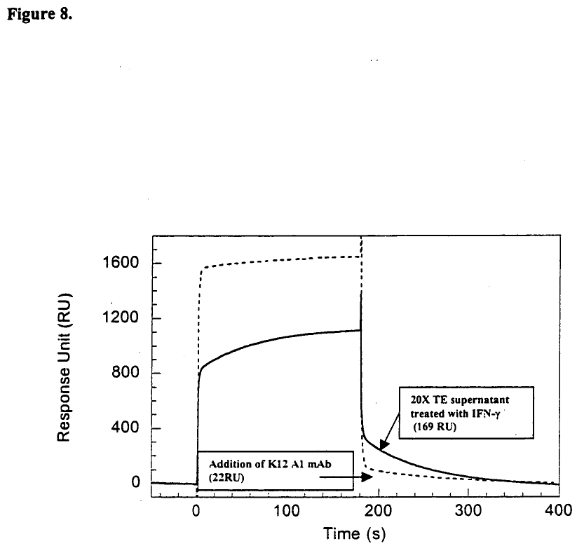

A third method of demonstrating K12 production by TE cells was to assay 20×concentrated TE supernatants for their ability to bind to immobilized K12-A1, hCD7-Ig or hCD8-Ig surfaces in Biacore assay (FIG. 7). FIG. 7A shows the reactivity of TE supernatants to K12-A1 surface and to hCD7-Ig surface but not to the hCD8-Ig surface. The higher affinity of the K12-A1 mab for K12 compared to the hCD7-Ig surface is seen as manifested by the faster off-rate of the captured supernatant K12 with hCD7-Ig compared to K12-A1 mab. FIG. 7B shows a representative experiment comparing the reactivity of 20×TE 750 supernatant from cells either incubated alone or with IFN-γ 500 IU/mL. Higher binding to K12-A1 on the Biacore chip is seen with IFN-γ TE 750 cell supernatant compared to untreated supernatant or media alone control. FIG. 7C shows the summary of three such experiments in which the binding of 20×IFN-γ treated TE 750 supernatant was compared to 10 untreated TE 750 supernatant. It was found that 500 IU/mL IFN-γ×48 hrs. significantly upregulated K12 production and secretion by TE 750 cells (p=0.02). In this setting, it was found that when hCD7-Ig was immobilized on the chip, the K12-A1 mab completely inhibited the binding of TE supernatant K12 to hCD7-Ig (FIG. 8).

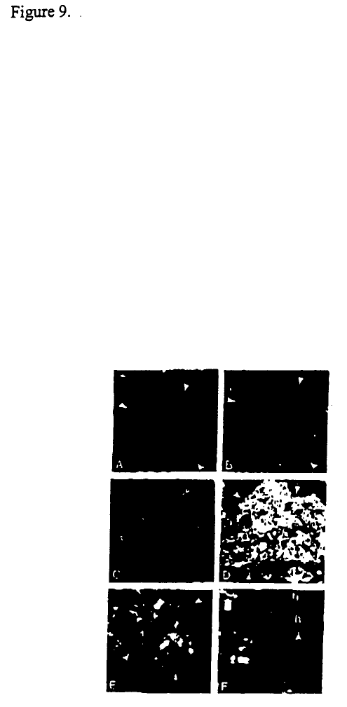

Expression of K12 in human TE cells in situ in thymus is regulated by IFN-γ. Finally, to determine the expression of K12 by human TE cells in situ in thymus, the K12-A1 mab was reacted with sections of thymus explants at 0 hours, 6 hrs., 18 hrs. and 24 hrs. following treatment with 500 IU/mL IFN-γ (FIG. 9). It was found that TE cells in fresh thymus tissue and TE cells in thymus tissue 6 hours after treatment with IFN-γ was not reactive with the K12-A1 mab. After 18 and 24 hrs in IFN-γ 500 IU/mL, TE cells became positive for K12 reactivity (FIGS. 9C and 9E, respectively).

All documents cited above are hereby incorporated in their entirety by reference.

What is claimed is:

1. A method of enhancing a vaccine-induced immune response comprising administering to a mammal receiving said vaccine an amount of a polypeptide that comprises the extracellular domain of K12, or portion of said extracellular domain that retains binding affinity for CD7, or mimetic thereof, sufficient to effect said enhancement.

2. The method according to claim 1 wherein said K12 is human K12.

3. The method according to claim 1 wherein said polypeptide is at least 95% homologous to the extracellular domain of human K12, or portion of said extracellular domain that retains binding affinity for CD7.

4. The method according to claim 1 wherein said polypeptide is soluble human K12.

5. The method according to claim 4 wherein said soluble K12 is present in a fusion protein.

6. The method according to claim 1 wherein said mammal is a human.

7. The method according to claim 1 wherein a nucleic acid that encodes said polypeptide is administered to said mammal under conditions such that said nucleic acid is expressed and said polypeptide is thereby produced.

8. The method according to claim 7 wherein said nucleic acid is present in a vector.

9. The method according to claim 7 wherein said nucleic acid is operably linked to a promoter.

10. A method of stimulating production of TFN-α, IL-2 and IFN-γ by TCRγδ cells and NK cells comprising administering to a mammal receiving a vaccine an amount of a polypeptide that comprises the extracellular domain of K12, or portion of said extracellular domain that retains binding affinity for CD7, or mimetic thereof, sufficient to effect said stimulation.

11. The method according to claim 10 wherein mammal is a human.

12. The method according to claim 10 wherein said K12 is human K12.

13. The method according to claim 10 wherein a nucleic acid that encodes said polypeptide is administered to said mammal under conditions such that said nucleic acid is expressed and said polypeptide is thereby produced.

14. The method according to claim 13 wherein said nucleic acid is present in a vector.

15. The method according to claim 13 wherein said nucleic acid is operably linked to a promoter.

16. A composition comprising a polypeptide that comprises the extracellular domain of K12, or portion of said extracellular domain that retains binding affinity for CD7, or mimetic thereof, and an antigen.

17. The composition according to claim 16 wherein said polypeptide is conjugated to said antigen.

18. A composition comprising a nucleic acid sequence encoding a polypeptide that comprises the extracellular domain of K12, or portion of said extracellular domain that retains binding affinity for CD7, or peptidomimetic thereof, and a nucleic acid sequence encoding an antigen.

19. A nucleic acid sequence encoding a fusion protein wherein said fusion protein comprises: i) the extracellular domain of K12, or portion of said extracellular domain that retains binding affinity for CD7, or peptidomimetic thereof, and ii) an antigen.
